(19) United States
(12) Patent Application Publication
(10) Pub. No.: US 2025/0169780 A1
(43) Pub. Date: May 29, 2025

(54) COMPUTER-IMPLEMENTED METHOD FOR OPERATING AN X-RAY DEVICE, AND X-RAY DEVICE

(71) Applicant: Siemens Healthineers AG, Forchheim (DE)

(72) Inventors: Philipp Roser, Erlangen (DE); Alois Regensburger, Poxdorf (DE)

(21) Appl. No.: 18/962,103

(22) Filed: Nov. 27, 2024

(30) Foreign Application Priority Data
Nov. 29, 2023 (DE) 10 2023 211 966.8

(51) Int. Cl. A61B 6/00; A61B 6/42

(52) U.S. Cl. CPC A61B 6/483; A61B 6/4275

(57) ABSTRACT

A computer-implemented method for operating an X-ray device for acquiring projection images of an acquisition area, from which a three-dimensional image dataset of the acquisition area is reconstructed. The X-ray device has an acquisition arrangement with an X-ray generator and an X-ray detector for receiving X-ray radiation of an X-ray field which has a central beam and which is transmitted by the X-ray generator. For acquiring projection images of different projection directions, the acquisition arrangement is moved rotationally to cover a projection angular range encompassing at least 360° about the acquisition area. An opacification device is used that includes an opacification pattern that includes a section opacifying the X-ray radiation with at least two opacifying areas and a section permeable to the X-ray radiation with at least two permeable areas separated by an opacifying area. The opacification device is arranged in the X-ray field between the X-ray generator and the acquisition area such that each voxel to be reconstructed in the acquisition area, and which is opacified in a partial area of the rotation, is scanned by non-opacified X-ray radiation in another partial area of the rotation, that includes at least substantially the same number of acquisition geometries and/or encompasses a 180° angular section with respect to the projection of the projection direction into the plane of rotation.

### CROSS REFERENCE TO RELATED APPLICATIONS

This application claims the benefit of DE 10 2023 211 966.8 filed on Nov. 29, 2023, which is hereby incorporated by reference in its entirety.

### FIELD

Embodiments relate to a computer-implemented method for operating an X-ray device for acquiring projection images of an acquisition area, from which a three-dimensional image dataset of the acquisition area is reconstructed.

### BACKGROUND

In X-ray imaging, for example in medical use, it is known for higher-dimensional image datasets to be reconstructed from lower-dimensional projection images, for example three-dimensional image datasets (for example as sectional image batches) from two-dimensional projection images. For example, an acquisition arrangement containing an X-ray generator and an X-ray detector may be moved about the acquisition area of an object to be acquired, in order to acquire the projection images from different projection directions. Here the X-ray generator moves on an acquisition trajectory, for example a circular path.

Whereas dedicated computed tomography devices are known, in which the X-ray generator and where appropriate the X-ray detector are moved in a gantry, it has already been proposed to implement computed-tomography-like acquisition procedures with other X-ray devices, for example X-ray devices with a C-arm, as are often employed in angiography laboratories. This type of acquisition is also referred to as “DynaCT”, or, since cone beam geometry is normally employed, “Cone Beam CT” (CBCT).

For example, when X-raying larger patients in “DynaCT”, problems may occur due to truncation artifacts and scattered radiation artifacts. As regards to truncation, it has been proposed to rotate through at least 360° minus the angular spacing between acquisition positions and to shift the isocenter of rotation in order to cover the patient as much as possible, for example by two isocenters shifted in respect of one another for partial rotations of at least 180°. It has further been proposed to shift the X-ray detector to one side of the central beam in each case and, with suitable collimation, to measure only one side in each case in order to capture the entire acquisition area. However, in many cases a large-area detector is employed, that may exacerbate scattered radiation problems.

As regards the scattered radiation, that is relevant for example for X-ray detectors with a large detection area, various measures to improve the image quality have also already been proposed. Using collimation, the X-ray field may be restricted to the size actually needed. Anti-scatter grids may be located adjacent to the X-ray detector, for example positioned on it, and include absorption elements directed at the X-ray generator, that may absorb scattered radiation components originating outside the path from the X-ray generator. Such absorption elements may for example be lamellae.

As regards to an image-based or computational improvement in the image quality as regards scattered radiation, it has been proposed to estimate the distribution of scattered radiation using software, for example by simulation, and to subtract it accordingly from the projection images. Optimization methods based on image quality metrics have also been proposed.

A relatively new variant for improving the image quality is known as “primary modulation”. In this case the primary radiation is modulated as regards the spatial frequencies by a primary modulator arranged between the X-ray generator and the acquisition area. Based on the hypothesis that low-frequency components dominate the scattered radiation even if high-frequency components are present in the incident primary radiation, and that scattered radiation is thus immune to high-frequency modulation, the primary radiation is marked by the imposition of high-frequency components, so that a separation between directly incident, attenuated primary radiation and scattered radiation becomes possible. To this end, reference is made to the article by L. Zhu et al., “Scatter correction method for X-Ray CT using primary modulation: Theory and preliminary results”, IEEE Trans. Med. Imaging 25 (12), pages 1573-1587, 2006, and “Scatter correction method for X-Ray CT using primary modulation: Phantom studies”, Med. Phys. 37 (2), pages 934 to 946, 2010.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide an improvement reducing losses in image quality caused by scattered radiation.

In a method is provided where an opacification device is used, that includes an opacification pattern, that includes a section opacifying the X-ray radiation with at least two opacifying areas and a section permeable to the X-ray radiation with at least two permeable areas separated by an opacifying area, and that is arranged in the X-ray field between the X-ray generator and the acquisition area such that each voxel to be reconstructed in the acquisition area, and that is opacified in a partial area of the rotation, is scanned by non-opacified X-ray radiation in another partial area of the rotation, that for example includes at least substantially the same number of acquisition geometries and/or includes a 180° angular section with respect to the projection of the projection direction into the plane of rotation.

The expanded angular range are used for the projection directions in a 360° scan (or even a scan encompassing more than) 360°, in order in partial angular ranges to opacify voxels of the acquisition area that are to be reconstructed from the X-ray radiation and thereby significantly to reduce the X-ray radiation in the acquisition area and thus also the scattered radiation. Nevertheless, each voxel is scanned, for example sufficiently for a reconstruction, since due to the rotation of the acquisition arrangement about the acquisition area and the corresponding embodiment of the opacification pattern for opacified voxels these are scanned in other angular positions of permeated, non-opacified X-ray radiation. If the rotation takes place at least substantially in a plane of rotation, the opacifying areas and the permeable areas therein may follow one another.

Because a large number of opacifying areas and permeable areas are selected, projection data is nevertheless obtained in each projection geometry, widely distributed in the acquisition area, so that a reliable data situation exists, for example also with regard to movements and the correction thereof, as well as the reconstruction, this having an advantageous effect on the image quality.

The opacification device is provided with the opacification pattern as a beam-blocking array on the X-ray generator side, in order to reduce the amount of scattered radiation in the projection images by reducing the illuminated volume. Because of the at least 360° rotation to be covered, potentially missing information in opacified detector areas is acquired in other projection geometries, for example at least substantially in opposing projection angles. Thus, the advantages of collimation and primary modulation are combined without any loss of information. An improved suppression of scattered radiation is achieved. Thus, a better image quality may be achieved.

Embodiments further provide an X-ray device with an acquisition arrangement that includes an X-ray generator and an X-ray detector is used. The acquisition and reconstruction operation may be controlled by a control device, that consequently may for example be configured for the performance of the described method. The X-ray device is for example an X-ray device with a C-arm, on which the X-ray generator and the X-ray detector are arranged opposite one another. The procedure may be employed when using a cone beam geometry, i.e. cone beam CT or dyna CT.

The acquisition of the projection images includes a rotation about the acquisition area to cover a projection angular range encompassing at least 360°, for example 358° or even more than 360°. If projection images are acquired at an angular spacing, a rotation of the acquisition arrangement about 360° minus the angular spacing is sufficient, since the first projection image need not be acquired anew. With an angular spacing of 2° this would result in a rotation of 358°, that is necessary for 360° coverage.

For example, the rotation may take place in a plane of rotation, consequently about a fixed axis of rotation, so that for each acquisition of a projection image a central beam (central projection beam) exists, that runs from the focus of the X-ray generator through the isocenter (point of intersection of axis of rotation and plane of rotation). Embodiments may also be applied to acquisition procedures in which the axis of rotation is not fixed, for example precesses or is otherwise subject to fluctuations, for example within a small area and/or about a nominal axis of rotation.

An opacification pattern of an opacification device, that is arranged between the X-ray generator and the acquisition area, includes an absorbent effect on some of the X-ray radiation of the X-ray field. Specifically, some of the X-ray field is masked in the opacifying areas. In this case it may be provided that an attenuation of the X-ray radiation by at least 80%, for example at least 90%, at least 95%, or at least 99%, occurs in the opacifying section of the opacification pattern and/or the permeable section includes an at least 5-fold, for example at least 10-fold, for example at least 20-fold, for example at least 50-fold, transmission compared to the opacifying section. This is in contrast to the primary modulation, where partially permeable grids or in general modulation structures are used for modulation.

In this case the opacification device, specifically the opacification pattern, is configured and arranged such that each voxel to be reconstructed is scanned sufficiently for the desired reconstruction. The opacifying section and the permeable section may be the same size in the X-ray field. Examinations have shown that during a rotation covering a 360° area, for example using a fixed axis of rotation, each projection beam is acquired twice. The configuration of the opacification pattern is now such that the projection beam may always permeate for at least one of these two times of acquisition, i.e. reaches the permeable section. Numerically too, it has been shown during operation of the present invention for inventive opacification patterns that any voxel to be reconstructed achieves 180° coverage by projection beams, as is required for the exact reconstruction. (This applies to cone beam geometry, as is known in principle, at least for the central projection plane, while for voxels lying outside this plane the projection onto the central projection plane, i.e. plane of rotation in which the central beam rotates, is considered in this respect).

The permeable section may be at least slightly larger than the opacifying section. This prevents information from being missing in the event that the system calibration deviates from the perfect acquisition trajectory. In addition, redundant information in overlapping areas may permit or improve movement compensation. For example, the size of the opacifying section may be 85 to 95% the size of the permeable section.

One of the main applications is with fixed axes of rotation, as already discussed. A corresponding, configuration in this connection provides that a circular path, by rotation of the acquisition arrangement about a fixed axis of rotation, is used as an acquisition trajectory of the X-ray generator, wherein the opacification pattern is divided into two sides by a central line running perpendicular to the plane of rotation and including the point of passage of the central beam, and is selected and arranged such that for each opacifying point on one side of the central beam there is a permeable point at the same distance on the other side of the central beam. The feature that for each opacifying point on one side of the central beam there is a permeable point at the same distance on the other side of the central beam, implements the feature whereby each voxel to be reconstructed in the acquisition area, and that is opacified in a partial area of the rotation, is scanned by non-opacified X-ray radiation in another partial area of the rotation, for example including an at least substantially identical number of acquisition geometries and/or encompassing a 180° angular section in respect of the projection of the projection direction into the plane of rotation. In other words, this embodiment relates to a computer-implemented method for operating an X-ray device for acquiring projection images of an acquisition area, from which a three-dimensional image dataset of the acquisition area is reconstructed, wherein the X-ray device includes an acquisition arrangement with an X-ray generator and an X-ray detector for receiving X-ray radiation of an X-ray field, that includes a central beam and that is transmitted by the X-ray generator, and for acquiring projection images of different projection directions the acquisition arrangement is moved along a circular path in a plane of rotation about a fixed axis of rotation rotating to cover a projection angular range encompassing at least 360° about the acquisition area, that is characterized in that an opacification device is used that includes an opacification pattern that includes a section opacifying the X-ray radiation with at least two opacifying areas and a section allowing the X-ray radiation to pass through with at least two permeable areas separated by an opacifying area, wherein the opacification pattern is divided into two sides by a central line running perpendicular to the plane of rotation and including the point of passage of the central beam, and is arranged in the X-ray field between the X-ray generator and the acquisition area such that for each opacifying point on one side of the central beam there is a permeable point at the same distance on the other side of the central beam.

For example, the central beam runs in the plane of rotation and the opacification pattern is perpendicular to the central beam. Along the central line through the central beam running perpendicular to the plane of rotation and through the opacification pattern the opacification device is expediently permeable, since there is no correspondingly opposing point here. In other words, the central line lies in the permeable section. Starting from the central line, the opacification pattern in the direction of the course of the plane of rotation may be divided into two sides, so that for each opacifying point of a side a permeable point is present at the same distance on the other side of the central line, and consequently there is an anti-symmetry in respect of the opacifying section.

These geometric relationships may also be described in respect of an angle of deviation from the central beam for a projection vector, for the cone beam geometry of the projection of the projection vector into the plane of rotation. The angles of deviation are positive on one side and negative on the other side. Opposing then means a mirroring of the projection vector, for the cone beam geometry of its sections projected into the plane of rotation, on the central beam. For each angle of deviation «, for which the opacification device is absorbent, an opposing angle of deviation-a exists, in which the opacification device is permeable.

As already mentioned, this may be understood to mean that the opacification pattern is anti-symmetrical in respect of a line of symmetry (central line) running perpendicular to the plane of rotation and through the central beam at least in respect of the opacifying section. Complete anti-symmetry means that the opacifying section and the permeable section are the same size, that, as already mentioned, is less preferable.

In an embodiment with a fixed axis of rotation it may be provided that the opacifying areas are designed as opacifying strips running perpendicular to the plane of rotation. Overall, this then results in a type of “array structure”. The width of the opacifying strips may here preferably be less than the width of the permeable strips. For example, it may be provided that the width of the opacifying strips in the direction of the plane of rotation (i.e. perpendicular to the central line) is 85 to 95% of the width of the permeable strips. In a specific example with reference to the above-defined angle of deviation, a permeable strip may for example cover an angular range of deviation of 1.05°, and an opacifying strip an angular range of deviation of 0.95°.

It may be provided that the width of the opacifying strips increases outward in a cone beam geometry in accordance with the law of inverse distance. This means that the widths of the opacifying strips and of the permeable strips may be a function of the covered detector gaps of the X-ray detector, in order to take account of cone beam geometries (or else fan beam geometries). For example, as the distance from the central beam or the central gap of the X-ray detector increases the widths become larger, in order to take account of the law of inverse distance.

The number of strips or, generally speaking, of opacifying and permeable areas, for example per side, may ultimately be chosen arbitrarily. It has however proven to be expedient if the opacification pattern on each side of the central beam includes at least two opacifying areas, for example strips, and at least two permeable areas, for example strips. Then despite the opacifying areas a nevertheless uniform coverage of the acquisition area is achieved in the different projection images.

Opacifying areas may be provided, for example perpendicular to the described opacifying strips, that run at least substantially parallel to the plane of rotation, in order further to reduce the primary radiation and thus also the scattered radiation, for example to configure the opacification pattern in the form of a grid. In this case the (then generally missing) information in respect of these opacifying areas, that run at least substantially parallel to the plane of rotation, may be estimated, for example by demodulation, or the acquisition trajectory may be adapted such that for example the acquisition arrangement is additionally tilted out of the nominal plane of rotation, in order to obtain full information from the acquisition area despite these opacifying areas. The width of such opacifying areas or strips, running parallel to the (nominal) plane of rotation, is then expediently less, for example significantly less, than that of the corresponding permeable areas/strips. In extreme cases in a configuration with fluctuation about a nominal plane of rotation a chessboard-like configuration of the opacification pattern may be used.

An embodiment may provide that the opacification device additionally also acts as a primary modulator, whose modulating property is used for scattered radiation correction of the projection images. For example, in the case of opacifying strips perpendicular to the plane of rotation partially permeable strips may be provided in the permeable section parallel to the plane of rotation, in order to achieve a modulation as regards the spatial frequencies of the primary radiation, that due to the demodulation, as described in the articles already cited in the introduction, may be removed again and may be used to separate scattered radiation. In this way the advantages of primary modulation and of partial opacification are therefore combined to produce a further improvement in the quality of the image dataset.

In a configuration it may be provided that in the areas of the X-ray detector covered by the opacifying section the radiation data describing scattered radiation is acquired and is used to determine a distribution of scattered radiation, that is used for scattered radiation correction. Consequently, in the areas of the X-ray detector opacified for direct X-ray radiation the scattered radiation is measured and may consequently be used for a scattered radiation model and/or scattered radiation correction. In this way, this consequently also results in an improved estimation of the (remaining) scattered radiation. For example, in a specific configuration a scattered radiation image for the entire corresponding projection image may be estimated on the basis of the scattered radiation data, in order to remove the remaining scattered radiation components, for example by subtraction, from the projection image. Corresponding procedures, in which a conclusion is drawn from measured scattered radiation as to the distribution of scattered radiation for a projection image, may consequently also be employed, since scattered radiation data may be collected here in larger sections of the X-ray detector and simultaneously with the acquisition of the projection images.

Compared to the primary radiation, opacified sections of the X-ray detector may also be used in other ways. Thus, it may be provided that between the acquisition area and the X-ray detector, for example directly in front of the X-ray detector, a scattered radiation grid may be used, that at the edge of the areas covered by the opacifying section includes at least one lamella that is enlarged compared to other lamellae of the scattered radiation grid. Scattered radiation grids are a known approach to reduce the scattered radiation incident on an X-ray detector. Absorption elements, also referred to as lamellae, are used here. However, these absorption elements should, on the other hand, in principle block less primary radiation. In practice it is however known for the lamellae of a scattered radiation grid also to be mapped in the projection image, so that they have to be subtracted. For example, so-called “supergrids” have been proposed, that have large lamellae that, although they attenuate the scattered radiation extremely well, also absorb an appreciable part of the direct X-ray radiation onto the X-ray detector. This modulation then has to be corrected. In this configuration of the present invention this problem may be largely eliminated, as large absorption elements (lamellae) may be employed directly on the borders of the opacification of the opacifying areas, in order to achieve excellent blocking of scattered radiation there. Thus even larger lamellae, that would result in modulation if a detector surface of the X-ray detector were used continuously for imaging, are employed expediently and advantageously without such problematic modulation, since opacified detector sections of known position are present.

It may further be provided that to determine a position of the opacification pattern to be used for the reconstruction this is detected in the projection images in an image-based manner. In other words, the position of the opacification pattern may be identified in an image-based manner in the projection images, so that even deviations, for example caused by inaccuracies in the calibration, do not result in errors in the reconstruction, since it is known precisely where X-ray radiation reaches the X-ray detector directly and where an opacification due to opacifying areas of the opacification device is present. Specifically, it may be provided that initially in the projection images the opacified image areas are segmented and are then compared to the known geometry of the opacification pattern in order to achieve a particularly high degree of accuracy.

The opacification device, that is pivotably mounted via an actuator, may be pivoted into the beam path before the projection images are acquired. For example, the opacification device may form part of a filter wheel. Thus, the opacification device may be selectively inserted into the beam path as required and removed when using other acquisition techniques, for example fluoroscopy.

In addition to the method, embodiments also provide an X-ray device, for example a C-arm-X-ray device. The X-ray device includes: an acquisition arrangement with an X-ray generator and an X-ray detector for receiving X-ray radiation of an X-ray field, that includes a central beam and that is transmitted by the X-ray generator, an opacification device, that includes an opacification pattern, that includes a section opacifying the X-ray radiation with at least two opacifying areas and a section permeable to the X-ray radiation with at least two permeable areas separated by an opacifying area, and is or may be arranged in the X-ray field between the X-ray generator and the acquisition area such that during an acquisition, in which the acquisition arrangement is moved rotationally about an acquisition area to cover a projection angular range encompassing at least 360° of projection images of the acquisition area, from which a three-dimensional image dataset of the acquisition area is reconstructed, each voxel to be reconstructed in the acquisition area, that is opacified in a partial area of the rotation, is scanned by non-opacified X-ray radiation in another partial area of the rotation, for example including an at least substantially identical number of acquisition geometries and/or encompassing a 180° angular section in respect of the projection of the projection direction into the plane of rotation, and a control device with an acquisition unit for controlling the acquisition of the projection images and a reconstruction unit for reconstructing the image dataset, taking into account the use of the opacification device.

The X-ray device may for example include a C-arm, on which the X-ray generator and the X-ray detector are arranged opposing one another. The C-arm may be rotatable about the axis of rotation of the rotation. In the case of an opacification device that may be pivoted into the beam path, the control device may be configured to control the corresponding actuator for pivoting the opacification device. In general, the control device may have at least one processor and at least one storage.

In respect of a fixed axis of rotation the X-ray device may include an acquisition arrangement with an X-ray generator and an X-ray detector for receiving X-ray radiation of an X-ray field that includes a central beam and that is transmitted by the X-ray generator, wherein the acquisition arrangement may be rotated, for example by the C-arm, about a fixed axis of rotation about an acquisition area such that the X-ray generator, for example the focus thereof, moves along a circular path, that lies in a plane of rotation, an opacification device, that includes an opacification pattern, that includes a section opacifying the X-ray radiation with at least two opacifying areas and a section permeable to the X-ray radiation with at least two permeable areas separated by an opacifying area, wherein the opacification pattern is divided into two sides by a central line running perpendicular to the plane of rotation and including the point of passage of the central beam, and is or may be arranged in the X-ray field between the X-ray generator and the acquisition area such that for each opacifying point on one side of the central beam there is a permeable point at the same distance on the other side of the central beam, and a control device with an acquisition unit for controlling the acquisition of projection images of an acquisition area along the circular path during a rotation to cover a projection angular range encompassing at least 360° in the case of an opacification device arranged between the X-ray generator and the acquisition area, and a reconstruction unit for reconstructing an image dataset from the projection images, taking account of the use of the opacification device.

The method may be implemented on the control device as a computer program that, if it is executed on the control device, causes the control device to execute the steps of the method. The computer program may be stored on an electronically readable data storage medium.

The use of the opacification pattern of the opacification device is completely independent of a collimation of the X-ray field by a collimation device (collimator) of the X-ray device. Such a collimator for example uses lateral lamellae to adapt the dimensions of the X-ray field to itself, whereas embodiments opacify within the X-ray field, i.e. for the area to be actually acquired, and exploit the fact that no loss of information nevertheless occurs due to the full rotation.

### BRIEF DESCRIPTION OF THE FIGURES

FIG. 1 depicts a schematic sketch of an X-ray device according to an embodiment.

FIG. 2 depicts an embodiment of an opacification pattern.

FIG. 3 depicts a representation of an acquisition geometry according to an embodiment.

FIG. 4 schematically depicts the illumination of an X-ray detector in different positions of an acquisition arrangement according to an embodiment.

FIG. 5 depicts an embodiment of an opacification pattern.

FIG. 6 depicts a further embodiment of an opacification pattern.

FIG. 7 depicts a flow chart of an embodiment of the method.

### DETAILED DESCRIPTION

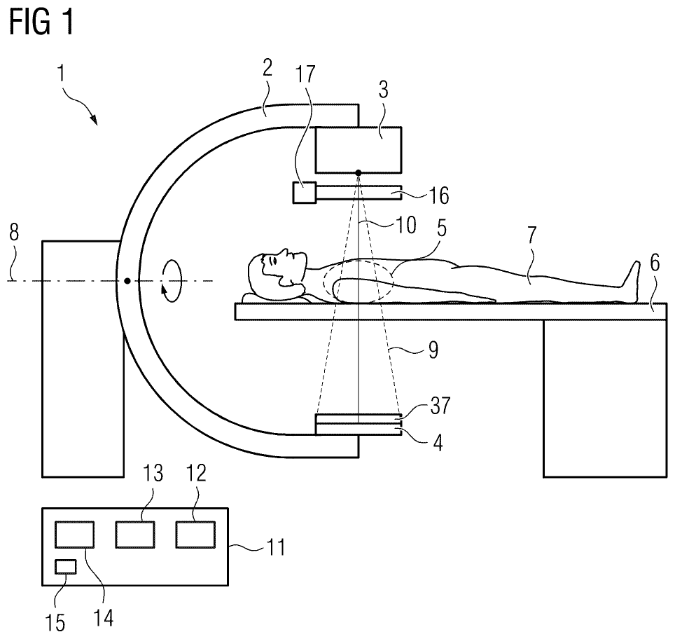

FIG. 1 depicts a schematic sketch of an X-ray device 1. This includes a C-arm 2, on which an X-ray generator 3 and an X-ray detector 4 are arranged opposing one another. The X-ray generator 3 and the X-ray detector 4 form an acquisition arrangement. The C-arm 2 permits a movement of this acquisition arrangement at least in different rotational degrees of freedom. In this way for example a rotation of the acquisition arrangement about an acquisition area 5 of an object under examination 7, here a patient, arranged on a patient couch 6 of the X-ray device 1, may take place, using a fixed axis of rotation 8 at least about a full 360° minus the angular spacing of the projection images, i.e. to cover a projection angular range encompassing at least 360°.

In this case an X-ray field 9 may be output to the X-ray detector 4 using the X-ray generator 3, in order to acquire projection images of the acquisition area 5 from different projection directions, that are defined by a central beam 10 of the X-ray field 9. In the present case a cone beam geometry is used.

The operation of the X-ray device 1 is controlled by a control device 11 indicated only schematically. The control device 11 has an acquisition unit 12 for controlling the acquisition operation and a reconstruction unit 13, by which three-dimensional image datasets may be reconstructed from projection images of different projection directions. Furthermore, a control unit 14 is provided for controlling further components of the X-ray device 1. Various information, for example including acquired projection data, may be stored at least temporarily in a storage 15.

The X-ray device 1 in the present case also includes an opacification device 16, that may optionally be brought into the beam path by an actuator 17, that may be controlled via the control unit 14, namely between the X-ray generator 3 and the examination area 5. In this case the opacification device 16 is moved together with the acquisition arrangement during a rotation, such that it remains stationary in respect of the X-ray generator 3 and the X-ray detector 4. For example, the opacification device 16, for example as part of a filter wheel, may be attached to the X-ray generator 3.

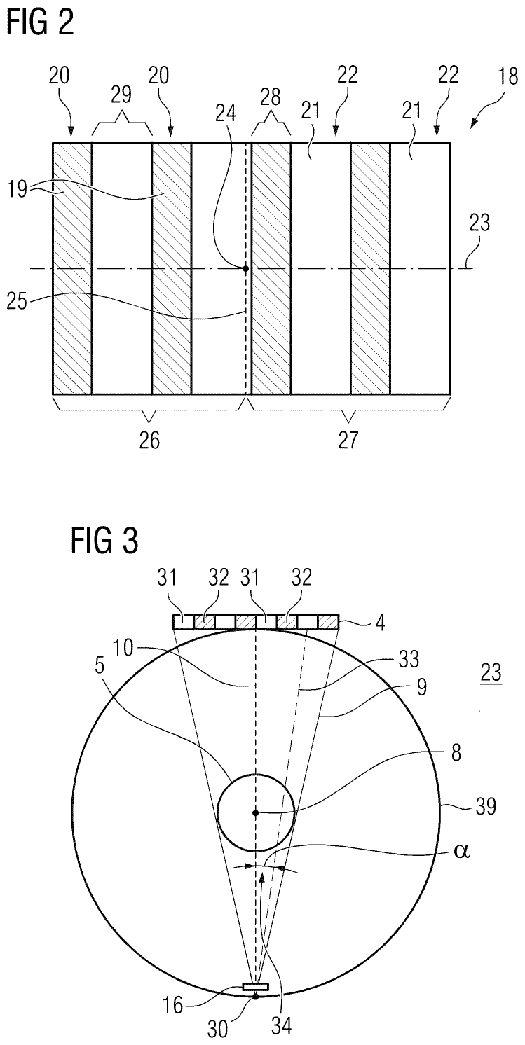

The opacification device 16 includes, in its section located in its swiveled-in position inside the X-ray field 9, an opacification pattern 18, for which a first embodiment is shown in greater detail in FIG. 2.

The opacification pattern 18 there includes, as an opacifying section, multiple opacifying areas 19 designed as strips 20 and multiple permeable areas 21, designed as strips 22, of a permeable section. In the present case the opacifying areas 19 absorb at least 95%, preferably at least 99%, of the X-ray radiation. Thus, the permeable areas 21 are at least ten times more transparent, for example at least twenty times more transparent, for the X-ray radiation than the opacifying areas 20. The permeable areas 21 are in this case each separated by an opacifying area 19.

For better orientation, the plane of rotation 23 of the fixed axis of rotation 8 is indicated, as is the point 24 at which the central beam 10 passes through the opacification pattern 18. Perpendicular to the plane of rotation 23 this therefore results in a central line 25 that as may be seen lies in the permeable section, as does the point 24. The central line 25 divides the opacification pattern 18 in the direction of the plane of rotation 23 into two sides 26, 27.

In respect of the opacifying section the opacification pattern 18 is now asymmetrical, so that for each opacifying point on one of the sides 26, 27 at the same distance from the central line 25 a permeable point exists on the other side 27, 26. In the present case the permeable section is larger than the opacifying section, for example therefore the width 28 of the opacifying strips 20 is less than the width 29 of the permeable strips 22.

FIG. 3 depicts how this configuration of the opacification pattern affects the X-ray detector 4. FIG. 3 depicts that the X-ray generator 3, of which only the focus 30 is shown here, moves as an acquisition trajectory on a circular path 39 because of the fixed axis of rotation 8. In this case the acquisition arrangement including X-ray generator 3 and X-ray detector 4 rotates together with the opacification device 16. The opacification pattern 18 in FIG. 2 extends in the plane of rotation 23 in the direction of a tangent to the circular path 39.

Because of the opacification pattern 18, areas 31 hit by direct X-ray radiation, that correspond to the permeable areas 21, and opacified areas 32, that correspond to the opacifying areas 19, are formed on the X-ray detector 4. Projection data of the projection images along the circular path 39 is acquired at different projection angles in the areas 31 of the X-ray detector 4, whereas in the opacified areas 32 of the X-ray detector 4 scattered radiation data is acquired, from which a scattered radiation image for the whole detector surface of the X-ray detector 4 may be derived, that may be employed by a correction unit of the control device 11 (not shown) or the reconstruction unit 13 for the correction of scattered radiation.

FIG. 3 also explains how the anti-symmetry in respect of the opacifying section of the opacification pattern 18 may be explained in another way. FIG. 3 depicts a projection beam 33, whose projection vector in the plane of rotation 23, that corresponds to the image plane in FIG. 3, where appropriate projected onto this, includes an angle of deviation 34 (α) to the central beam 10. This angle of deviation 34 is specified toward side 26 positively, and toward side 27 negatively. In the opacification pattern 18 there is now for each angle of deviation 34 (α) for which the opacification pattern 18 is absorbent, an opposing angle of deviation 34 (−α) for which the opacification pattern 18 is permeable.

If projection images are acquired along the circular path 39 during a rotation in different projection directions to cover a projection angular range encompassing at least 360°, it is shown that because of the special configuration of the opacification pattern 18 despite the opacifying areas 19 each voxel to be reconstructed is sufficiently scanned in the acquisition area 5, for example at least in respect of the projection into the plane of rotation 23 from an angular section encompassing at least 180°.

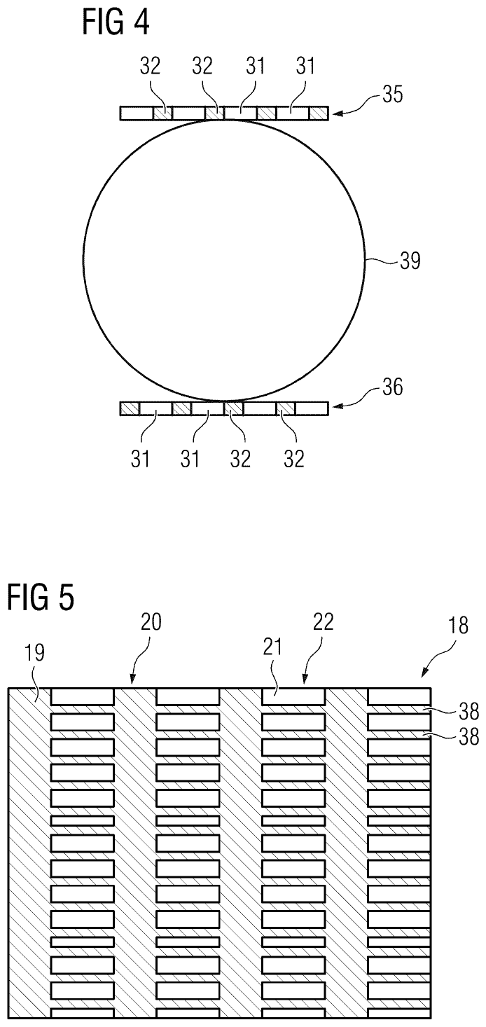

The reason for this is that different areas are opacified in different positions of the acquisition arrangement, as is schematically indicated by FIG. 4. This schematically depicts two opposing positions 35, 36, offset by 180°, of the X-ray detector 4. The respective position of the opacified areas 32 and of the non-opacified areas 31 that may be directly illuminated by the primary X-ray radiation illustrates the different sections acquired in projection images. It may be shown mathematically and numerically that with a revolution covering 360° each projection beam 33 occurs twice and at least once is not opacified by an opacifying area 19.

In order to ascertain the precise position of the opacification pattern 18 for the reconstruction of an image dataset from the projection images, the position is determined in an image-based manner from the projection images on the basis of the opacified areas 32.

Returning to FIG. 1, the X-ray device 1 also includes an anti-scatter grid 37 connected upstream of the X-ray detector 4, for example arranged on it, whose structure in the present case is also adapted to the configuration of the opacification pattern 18 in order to achieve an improved reduction in scattered radiation. To this end, large lamellae, i.e. absorption elements, are employed at the edge of the opacified areas 32, that would be mapped into the projection image in areas used for imaging, but may be employed in the opacified areas 32 without any problems.

FIG. 5 depicts a second embodiment of an opacification pattern 18, consequently of an opacification device 16. The opacification pattern 18 in FIG. 5 further includes the opacifying strips 20 as opacifying areas 19 and the permeable strips 22 as permeable areas 21. However, this has been supplemented by partially permeable strips 38, running orthogonally to the strips 20, 22, that serve for the high-frequency spatial frequency modulation of the primary X-ray radiation, so that at the X-ray detector 4 this may be separated from the scattered radiation, as known from primary modulation. The modulation may be removed again by demodulation.

The strips 23 may be configured as absorbent parts of the opacifying section and to use opacification, for example demodulation, to recover missing information. It is also conceivable to deviate at least slightly from the nominal plane of rotation by pivoting or tilting, for example sinusoidally, in order to measure full information.

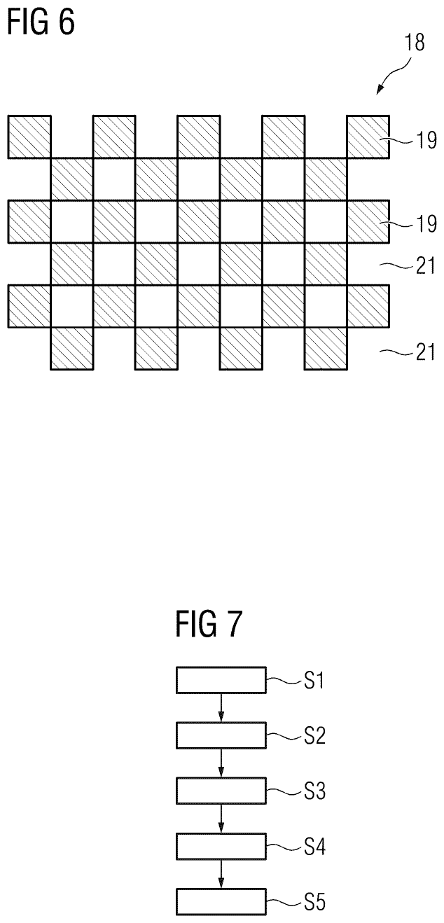

In such a context of acquisition trajectories deviating from a fixed axis of rotation 8, a third embodiment of an opacification pattern 18, as shown in FIG. 6, is also conceivable, in which opacifying areas 19 and permeable areas 21 are arranged in the manner of a chessboard.

FIG. 7 depicts a flow chart of an embodiment of the method, as may be performed by the control device 11. In this case in a step S1 the control unit 14 is initially used to control the actuator 17, in order to bring the opacification device 16 into the beam path of the X-ray field 9. In a step S2 the acquisition unit 12 is used to move the acquisition arrangement, for example using the circular path 39, rotationally about the acquisition area 5 and projection images and scattered radiation data are acquired in different projection directions, here projection angles. In this case the rotation takes place to cover a projection angular range encompassing at least 360°.

In a step S3 the scattered radiation data from the opacified areas 32 is used to determine a scattered radiation image for each projection image, that is used for the correction thereof as regards the scattered radiation. This may for example take place in a dedicated correction unit, not shown here in greater detail, or else already in the reconstruction unit 13.

In this, in a step S4, the positions of the opacification pattern 18 and thus of the opacification device 16 are also ascertained in an image-based manner for the individual projection images. Thus, in step S5 a high-quality reconstruction of an image dataset from the projection images may take place in the reconstruction unit 13.

Although the invention has been illustrated and described in detail by the preferred exemplary embodiment, the invention is not restricted by the disclosed examples and other variations may be derived therefrom by the person skilled in the art, without departing from the scope of protection of the invention.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A system for operating an X-ray device for acquiring projection images of an acquisition area from which a three-dimensional image dataset of the acquisition area is reconstructed, the system comprising:
an acquisition arrangement comprising an X-ray generator and an X-ray detector configured to receive X-ray radiation of an X-ray field that includes a central beam and which is transmitted by the X-ray generator, the X-ray detector configured for acquiring projection images of different projection directions, wherein the X-ray generator and X-ray detector are moved rotationally to cover a projection angular range encompassing at least 360° about the acquisition area; and
an opacification device comprising an opacification pattern that includes a opacifying section for opacifying the X-ray radiation with at least two opacifying areas and a section permeable to the X-ray radiation with at least two permeable areas separated by an opacifying area, wherein the opacification pattern is arranged in the X-ray field between the X-ray generator and the acquisition area such that each voxel to be reconstructed in the acquisition area that is opacified in a partial area of the rotation, is scanned by non-opacified X-ray radiation in a second partial area of the rotation, wherein the second partial area comprises at least substantially a same number of acquisition geometries and/or encompasses a 180° angular section with respect to the projection of the projection direction into a plane of rotation.

2. The system of claim 1, wherein an attenuation of the X-ray radiation by at least 80% occurs in the opacifying section of the opacification pattern and/or the permeable section has an at least 5-fold transmission compared to the opacifying section.

3. The system of claim 1, wherein the opacifying section is smaller than the permeable section.

4. The system of claim 1, wherein a circular path, by rotation of acquisition arrangement about a fixed axis of rotation, is used as an acquisition trajectory of the X-ray generator, wherein the opacification pattern is divided into two sides by a central line running perpendicular to the plane of rotation and including a point of passage of the central beam, and is selected and arranged such that for each opacifying point on one side of the central beam there is a permeable point at the same distance on the other side of the central beam.

5. The system of claim 4, wherein the at least two opacifying areas are configured as opacifying strips running perpendicular to the plane of rotation.

6. The system of claim 5, wherein a width of the opacifying strips is less in the direction of the plane of rotation than a width of the corresponding permeable strips of the permeable section and/or the width of the opacifying strips increases outward in a cone beam geometry in accordance with the law of inverse distance.

7. The system of claim 6, wherein the opacification pattern on each side of the central beam includes at least two opacifying areas and at least two permeable areas.

8. The system of claim 1, wherein the opacification device additionally also works as a primary modulator, whose modulating property is used for scattered radiation correction of the projection images.

9. The system of claim 1, wherein for the at least two opacified areas of the X-ray detector covered by the opacifying section, radiation data describing scattered radiation is acquired and is used to determine a distribution of scattered radiation, which is used for the scattered radiation correction.

10. The system of claim 1, wherein between the acquisition area and the X-ray detector, a scattered radiation grid is used that at an edge of the opacified areas covered by the opacifying section has at least one lamella which is enlarged compared to other lamellae of the scattered radiation grid.

11. The system of claim 1, wherein to determine a position of the opacification pattern to be used for the reconstruction, the opacification pattern is detected in an image-based manner in the projection images.

12. The system of claim 1, wherein the pivotally mounted opacification device is pivoted into a beam path via an actuator before a start of the acquisition of the projection images.

13. A method comprising:
controlling an actuator by a control unit to bring an opacification device into a beam path of an x-ray field;
moving an acquisition arrangement rotationally about an acquisition area in order to acquire, by the acquisition arrangement, projection images and scatter radiation data in different projection directions, wherein the acquisition arrangement comprises an X-ray generator and an X-ray detector for receiving X-ray radiation of an X-ray field which has a central beam and which is transmitted by the X-ray generator;
determining a scatter radiation image for each projection image from the scattered radiation data;
ascertaining positions of the opacification device for the projection images; and
reconstructing an image data set from the projection images.

14. An X-ray device comprising:
an acquisition arrangement with an X-ray generator and an X-ray detector for receiving X-ray radiation of an X-ray field which has a central beam and which is transmitted by the X-ray generator;
an opacification device comprising an opacification pattern that includes a section opacifying the X-ray radiation with at least two opacifying areas and a section permeable to the X-ray radiation with at least two permeable areas separated by an opacifying area, and is or may be arranged in the X-ray field between the X-ray generator and an acquisition area such that during an acquisition, in which the acquisition arrangement is moved rotationally about an acquisition area to cover a projection angular range encompassing at least 360° of projection images of the acquisition area, from which a three-dimensional image dataset of the acquisition area is reconstructed, each voxel to be reconstructed in the acquisition area that is opacified in a partial area of the rotation, is scanned by non-opacified X-ray radiation in another partial area of the rotation; and
a control device comprising an acquisition unit for controlling the acquisition of the projection images and a reconstruction unit for reconstructing the image dataset, taking into account the use of the opacification device.
